(19) United States
(12) Patent Application Publication
(10) Pub. No.: US 2023/0145748 A1
(43) Pub. Date: May 11, 2023

(54) DIGITAL MAT SYSTEM FOR MANAGEMENT AND ANALYSIS OF USER'S COGNITIVE AND PHYSICAL ABILITY

(71) Applicant: AHA Consulting Co., Ltd., Seoul (KR)

(72) Inventor: Young Hee RO, Gyeonggi-do (KR)

(73) Assignee: AHA Consulting Co., Ltd., Seoul (KR)

(21) Appl. No.: 17/523,158

(22) Filed: Nov. 10, 2021

(51) Int. Cl. A63B 21/00; A63B 23/04; A63B 24/00; A63B 71/06; A61B 5/16; A61B 5/00

(52) U.S. Cl. CPC A63B 21/4037; A63B 23/0458; A63B 24/0006; A63B 24/0062; A63B 71/0622; A61B 5/165; A61B 5/4088; A61B 5/6892; A63B 2024/0015; A63B 2071/065; A63B 2225/50; A63B 2225/74; A63B 2230/625

(57) ABSTRACT

Provided is a digital mat system for managing and analyzing cognitive and physical abilities of a user that is capable of managing and analyzing individual cognitive and physical abilities of a user (particularly the elderly) and providing a customized solution that matches individual abilities, thereby assisting in preventing geriatric diseases, such as falling and dementia.

### FIELD AND BACKGROUND OF THE INVENTION

The present invention relates to a digital mat system for managing and analyzing cognitive and physical abilities of a user that is capable of managing and analyzing individual cognitive and physical abilities of a user (particularly the elderly) and providing a customized solution that matches individual abilities, thereby assisting in preventing geriatric affliction, such as falling and dementia.

As the birth rate and mortality rate of some newborns decrease and the average life expectancy increases, the proportion of the elderly population is increasing.

A common disease that occurs in the elderly living in such an aging society is dementia.

While the social burden due to the aging population is continuously increasing, management of the elderly with dementia, who are very dependent on family and society, has become a more serious social problem as the family support function is weakening due to an increasing number of nuclear-families.

In Japan, the square-stepping exercise was developed to prevent and improve dementia. The square-stepping-exercise was designed in consideration of the mechanism of falling by tripping and the characteristics of ladder training of an athlete program and was born based on experience through practical research over a long period of time.

The square-stepping-exercise is a form of exercise in which continuous movements of forward, backward, left and right, and diagonal directions are performed in a certain order on a felt mat of which a rectangular surface having a width of 100 cm and a length of 250 cm is divided into 40 squares having a size of 25 cm, and has been reported to be useful in improving even cognitive functions as well as having effects related to physical functions such as fall prevention and physical strength improvement.

However, the square-stepping-exercise from Japan is provided to perform a gait exercise according to a certain rule, and there is a difficulty in generating objective data for managing and analyzing individual cognitive and physical abilities, and the square-stepping-exercise may not aid in improving individual cognitive and physical abilities.

### SUMMARY OF THE INVENTION

The present invention is directed to providing a digital mat system for managing and analyzing cognitive and physical abilities of a user that is capable of providing a square-stepping exercise that is optimized for an individual. The present invention provides a health care service optimized for an individual through the digital mat system.

According to an aspect of the present invention, there is provided a digital mat system for managing and analyzing cognitive and physical abilities of a user, the digital mat system including a square-stepping exercise mat for providing a sensing result on a square-stepping exercise; and a user health care device for providing a user with health information, wherein the square-stepping exercise mat includes: a communication unit configured to receive a first exercise rule optimized for the user with respect to the square-stepping exercise subjected to signal processing; and a pad formed in a rectangular shape having a width of 100 cm and a length of 250 cm and divided into 40 squares each having one side measuring 25 cm, the pad includes a first layer and a second layer formed of a material capable of absorbing an internal shock and preventing slipping; a control layer and a sensor layer formed between the first layer and the second layer; a light emitting diode (LED) layer for sequentially illuminating the squares corresponding to the first exercise rule; and a third layer for protecting the LED layer and transmitting LED light, further comprising the control layer configured to control an operation of the sensor layer and the LED layer, and the sensor layer configured to sense the square which the user steps on according to the first exercise rule.

The user health care device may include a controller, the controller including: a user personal information input unit configured to provide a user interface to receive personal information of the user; an information processing unit configured to select the first exercise rule according to health information of the user generated or updated on the basis of the personal information and the exercise information of the user; and an exercise mat control unit configured to transmit the first exercise rule to the square-stepping exercise mat and receive the exercise information through short-distance communication.

The user health care device may include: a motion sensor module provided at an upper side to detect an exercise motion when the user performs an exercise in an area other than the square-stepping exercise mat and transmit the exercise motion to the controller; a hologram stage provided at a lower side of the motion sensor module to output a correct exercise posture to the user in a hologram such that the user follows the exercise posture; and a hologram generating module provided on a lower portion of the hologram stage to generate a hologram on the hologram stage, wherein the controller may be installed in the hologram generating module.

The personal information may be periodically updated on the basis of the exercise information of the user.

The personal information may include information for identifying the user and information about a health status of the user, and the information about the health status of the user may include at least one of a dementia test result (Korea-Mini Mental State Examination: K-MMSE) of the user, information about a cognitive function of the user, a measurement of daily living fitness of the user, a degree of a risk of the user falling, a brain age of the user, a longevity of the user, and a happiness index of the user.

The user health care device may be formed using a cloud system, and the digital mat system further include an application for providing a user interface between the user health care device and the square-stepping exercise mat.

The information processing unit may be configured to: analyze information about a health status of the user included in the personal information to calculate a brain health age capable of reflecting a brain status and a physical status; and reflect the exercise information in the brain health age to generate the health information.

### BRIEF DESCRIPTION OF THE SEVERAL VIEWS OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram illustrating a digital mat system for managing and analyzing cognition and physical abilities of a user according to an embodiment of the present invention;

FIG. 2 is a detailed block diagram illustrating a digital mat system for managing and analyzing cognitive and physical ability of a user according to an embodiment of the present invention;

FIG. 3 is a flowchart showing an operating method of a square-stepping exercise system according to an embodiment of the present invention;

FIG. 4 is a diagram illustrating extraction of lower body joint data of a user by a digital mat system for managing and analyzing cognitive and physical abilities of a user according to an embodiment of the present invention;

FIG. 5 is a diagram illustrating extraction of a shoulder inclination of a user by a digital mat system for managing and analyzing cognitive and physical abilities of a user according to an embodiment of the present invention;

FIG. 6 is a diagram illustrating a square-stepping exercise rule according to an embodiment of the present invention;

FIG. 7 is a diagram illustrating a square-stepping exercise mat (SSMT) according to an embodiment of the present invention;

FIG. 8 is a diagram illustrating a method of analyzing and evaluating a health status of a user according to an embodiment of the present invention; and

FIG. 9 is a diagram illustrating a system for providing a health care service in a cloud environment according to an embodiment of the present invention.

### DESCRIPTION OF SPECIFIC EMBODIMENTS OF THE INVENTION

The embodiments to be described in the present invention are provided so that the present invention may be thorough and complete and may fully convey the scope of the invention to those skilled in the art. The embodiments to be described in the present invention may be realized in various forms, and the scope of the present invention is not limited to such embodiments.

The scope of the present invention is to cover all modifications, equivalents, and alternatives falling within the spirit and scope of the present invention.

In the description of the drawings, the same drawing reference numerals will be understood to refer to the same elements, features, and structures. In the accompanying drawings, the shape and size of each component shown in the drawings can be exaggerated for clarity of explanation.

Terms used herein are used for aiding in the description and understanding of the embodiments and are not intended to limit the scope and spirit of the present invention. It should be understood that the singular forms “a” and “an” also include the plural forms unless the context clearly dictates otherwise. The terms “comprises,” “comprising,” “includes,” and/or “including,” when used herein, specify the presence of stated features, integers, steps, operations, elements, components and/or groups thereof and do not preclude the presence or addition of one or more other features, integers, steps, operations, elements, components, and/or groups thereof. In connection with assigning reference numerals to elements in the drawings, the same reference numerals are used for designating the same elements throughout the specification, and the term “and/or” includes any one or combinations of the associated listed items. It should be understood that, although the terms “first,” “second,” etc. may be used herein to describe various features, these features are not limited by these terms. These terms are only used for distinguishing one feature from another. In the following description, when a first feature is referred to as being connected, coupled, or connecting to a second feature, it does not preclude the presence of a third feature between the first feature and the second feature.

Unless otherwise defined, all terms (including technical and scientific terms) used herein have the same meaning as commonly understood by one of ordinary skill in the art to which this invention belongs. It should be further understood that terms, such as those defined in commonly used dictionaries, should not be interpreted in an idealized or overly formal sense unless expressly specifically defined herein.

Hereinafter, embodiments will be described with reference to the accompanying drawings.

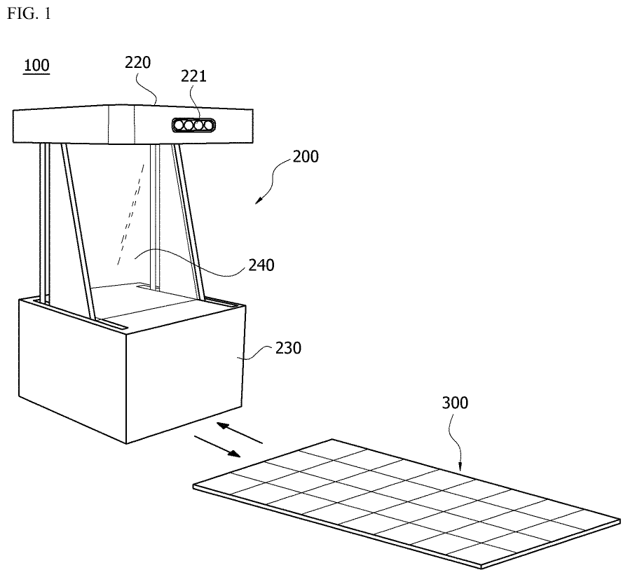

FIG. 1 is a block diagram illustrating a system for providing a health care service including a square-stepping exercise mat according to an embodiment of the present invention.

Referring to FIG. 1, the system 100 for providing a health care service according to the embodiment of the present invention includes a user health care device 200 and a square-stepping exercise mat (SSMT) 300.

The user health care device 200 generates user health information corresponding to a result of a user performing a square-stepping exercise on the SSMT 300 and provides the generated user health information.

The user health care device 200 includes a controller 210, and the controller 210 may include a user personal information input unit 211, an information processing unit 212, an exercise mat control unit 213, a user health information storage unit 214, and a user health information output unit 215.

The SSMT 300 is provided as a rectangular pad having a width of 100 cm and a length of 250 cm, and including 40 squares each having one side measuring 25 cm. In the following description, each of the square areas of the square-stepping exercise mat 300 is described as a square.

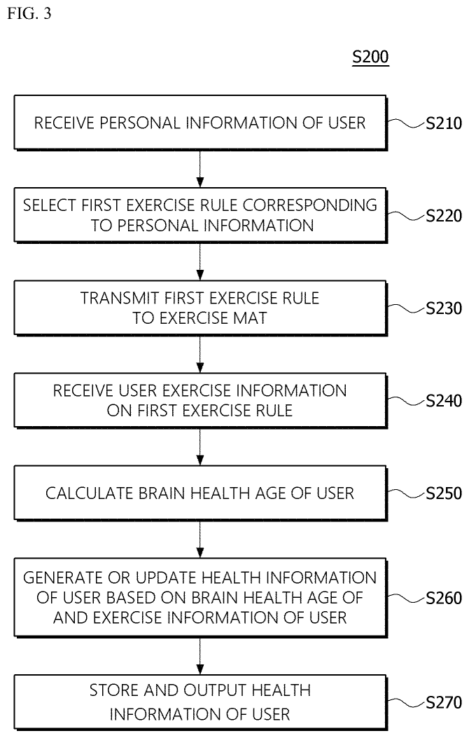

FIG. 3 is a flowchart showing an operating method of the system for providing a health care service including the SSMT according to the embodiment of the present invention.

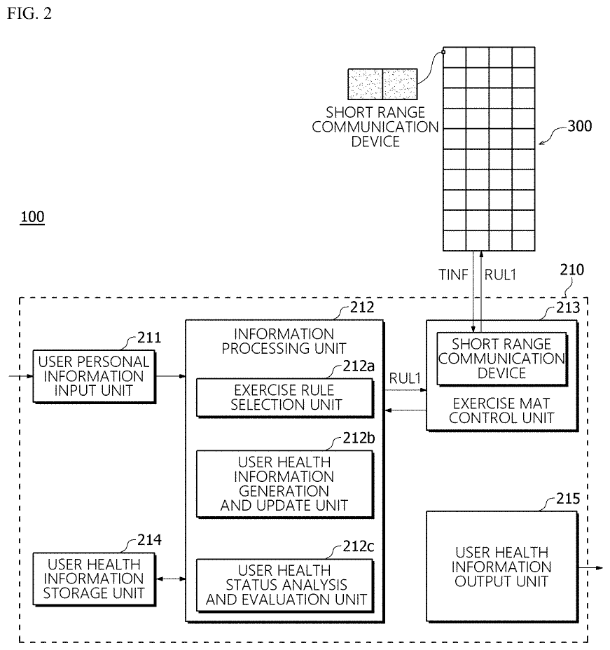

The system 100 for providing a health care service shown in FIGS. 1 and 2 may operate in the method S200 shown in FIG. 3 or other methods.

Referring to FIGS. 1 and 2, the user personal information input unit 211 provides a user interface to receive personal information input from a user (S210). The personal information may include a name, age, gender, and health status.

The information about the health status of the user may include a dementia test result (Korea-Mini Mental State Examination: K-MMSE) of the user, information about cognitive functioning of the user, a measurement of daily living fitness of the user, a degree of a risk of the user falling, a brain age of the user, a longevity of the user, and a happiness index of the user. When needed, the personal information may include information updated about the health status of the user.

For example, update information about the health status of the user after participating in the square-stepping exercise may be included as the personal information. The above-described update may be performed periodically. Alternatively, the personal information may include only a user identifier and/or password at normal times.

The information processing unit 212 receives the personal information, and exercise information, which is related to a result of a user performing a first exercise rule RUL1 on the SSMT, and analyzes and evaluates the health status of the user on the basis of the personal information and the exercise information to generate or update health information of the user and provide an exercise rule optimized for the user on the basis of the health information of the user.

To this end, the information processing unit 212 may include an exercise rule selection unit 212a, a user health information generation and update unit 212b, and a user health status analysis and evaluation unit 212c.

The exercise rule selection unit 212a selects the first exercise rule RUL1 corresponding to personal information, that is, optimized for each user (S220).

The first exercise rule RUL1 may be one of a plurality of square-stepping exercise rules. The square-stepping exercise is an exercise that ensures effectiveness and safety through practical research on thousands of elderly people over a period of ten years.

The square-stepping exercise rule refers to a rule about the order of performing continuous movements of forward, backward, left and right, and diagonal directions for 40 squares partitioned on the SSMT 300.

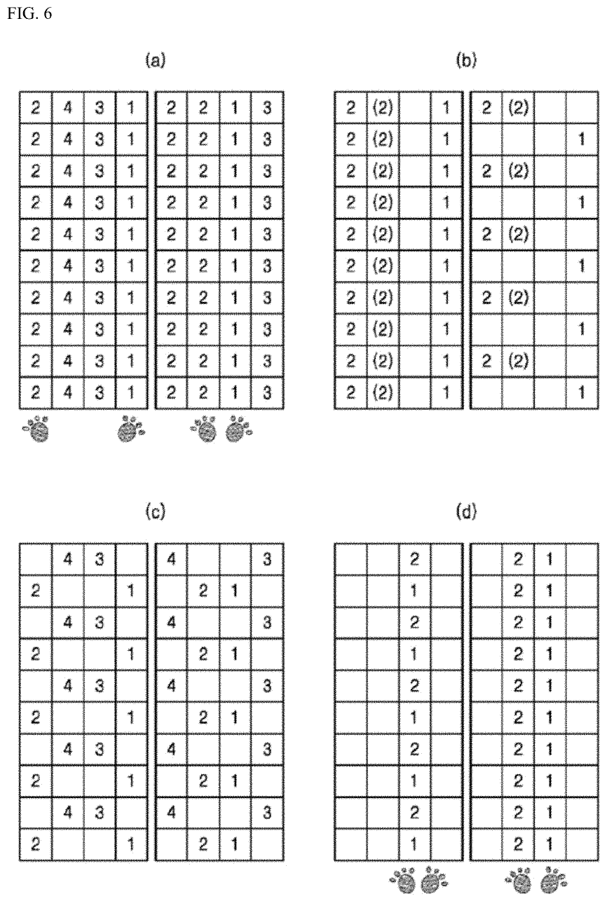

For example, referring to FIG. 6 showing square-stepping exercise rules according to an embodiment of the present invention, the exercise rule shown on the left side of FIG. 6A represents an exercise rule of stepping on a square 1 located on the rightmost among the lowest-stage four squares in the SSMT 300 first, and then stepping on a square 2 located on the leftmost, and sequentially stepping on a square 3 and a square 4, followed by proceeding to the next-stage squares in the same manner, repeating up to the uppermost stage.

Alternatively, the exercise rule shown on the right side of FIG. 6D represents an exercise rule that proceeds forward by sequentially stepping on the central two squares 1 and 2.

As described above, the first exercise rule RUL1 represents an optimal exercise rule based on health information of a user.

For example, when a user has a cognitive function that is normal and a low risk of falling on the basis of health information of the user, the exercise rule of FIG. 6A may be selected as the first exercise rule RUL1.

On the other hand, as for a user with a cognitive function that is normal but with a relatively high risk of falling, the exercise rule of FIG. 6B may be selected, and as for a user with mild cognitive impairment, the exercise rule of FIG. 6C may be selected as the first exercise rule RUL1. Alternatively, as for a user with mild cognitive impairment and a relatively high risk of falling, the exercise rule of FIG. 6D may be selected as the first exercise rule RUL1.

The exercise rule selection unit 211a may separately store information about exercise rules that each correspond to one piece of user health information HINF or may receive the information about exercise rules from a separate storage device. When the first exercise rule RUL1 optimized for a user cannot be selected because the user uses the system 100 for providing a health care service for the first time, the exercise rule selection unit 211a may select a default exercise rule set for the age, etc. included in the personal information as the first exercise rule RUL1.

The health information of the user may be generated not only from the cognitive function and the risk of falling, but also on the basis of a result of evaluating and analyzing the health status of the user based on a dementia test result of the user, a measurement of daily living fitness of the user, a degree of a risk of the user falling, a longevity of the user, and a happiness index of the user, together with a square-stepping exercise result to be described below.

The exercise mat control unit 213 performs predetermined signal processing on the first exercise rule RUL1 and transmits the signal-processed first exercise rule RUL1 to the SSMT 300 (S230).

The exercise mat control unit 213 may communicate with the SSMT 300 through a short-range communication device, for example, Bluetooth, etc. The SSMT 300 turns on LEDs of relevant areas (in units of squares) in an order corresponding to the first exercise rule RUL1. For example, when the first exercise rule RUL1 is the exercise rule shown on the left side of FIG. 6D, the SSMT 300 first turns on and turns off the square 1 located in the second column from the right on the lowermost stage, and turns on and turns off the square 2 of the same column on the next stage, which repeats the operation to the upper most stage in the same manner, so that the exercise rule to be executed may be visually presented to the user.

On the other hand, the user health care device 200 may not only have a function of providing the user health information corresponding to the result of performing the square-stepping exercise but also have a function that, when performing an exercise having standardized motions in a place other than the SSMT 300, such as yoga or squat, outputs a correct exercise posture to the user through a hologram to induce the user to follow the correct motion.

To this end, the user health care device 200 may include a motion sensor module 220 provided on the upper side to detect the motion of a user, a hologram stage 230 provided at the lower side of the motion sensor module 220 to output a correct exercise posture to the user in a hologram for the user to follow the exercise posture, and a hologram generating module 240 provided on a lower portion of the hologram stage 230 to generate a hologram on the hologram stage 230 as shown in FIG. 1.

The motion sensor module 220 includes a motion sensor 221. The motion sensor 221 generates joint data of upper and lower bodies of the user when the user takes an exercise motion. The generated joint data of the upper and lower bodies is transmitted to the controller 210 such that the joint data is compared with a normal motion output from the hologram stage 230 to identify whether there is a motion error.

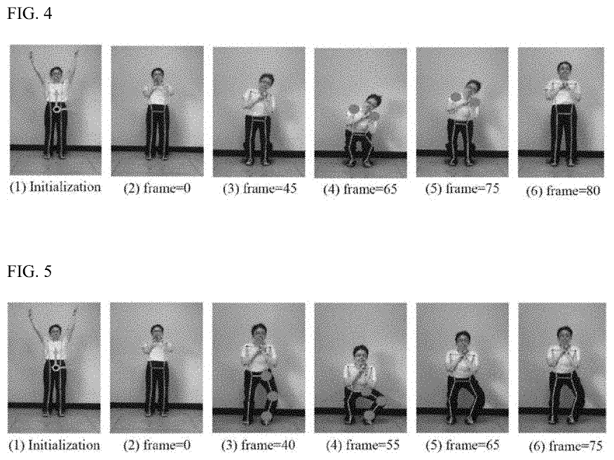

For example, during a squat exercise, as in FIGS. 4 and 5, whether a normal motion or an abnormal motion is performed may be identified by the knee angle and the waist angle of the user, and whether a normal motion or an abnormal motion is performed may be generally identified by the left and right inclination of the shoulder of the user. When a determination result is derived from the controller 210, the result value may be transmitted to a mobile phone of the corresponding user.

The controller 210 may be installed inside a casing of the hologram generating module 240.

A microphone and a speaker may be installed in the user health care device 200. The microphone may recognize speech of a user to select an exercise course (e.g., yoga, squat, etc.), and the speaker may output speech to guide the user to the correct posture when the posture of the user does not match a character posture of the hologram.

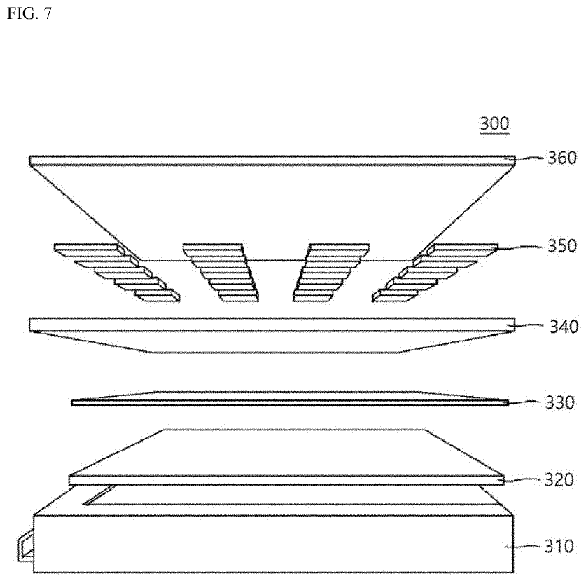

FIG. 7 is a diagram illustrating an example of a SSMT according to an embodiment of the present invention. Referring to FIG. 7, the square-stepping exercise mat 300 may include a first layer 310, a control layer 320, a sensing layer 330, a second layer 340, an LED layer 350, and a third layer 360.

The first layer 310 and the second layer 340 may be formed of a material having an anti-slip function. The first layer 310 and the second layer 340 may be manufactured using, for example, ethylene-vinyl acetate (EVA) copolymer or polyvinyl chloride (PVC) having softness, excellent flexibility, strength for internal impact, and excellent absorption.

The control layer 320 controls operations of the sensing layer 330 and the

LED layer 350. That is, the control layer 320 may process sensing information regarding a square of the sensing layer 330 stepped on by the user as exercise information with respect to the coordinates and time (sequence) of the corresponding square, and turn on corresponding LEDs of the LED layers 350 according to the first exercise rule RUL1 subjected to signal processing.

Unlike FIG. 7, the control layer 320 may be provided as a part of the sensing layer 330 and/or the LED layer 350. In addition, the control layer 320 may be controlled to cause sound effects to be output when the user steps on a corresponding square, for example, may provide the exercise mat control unit 213 with the corresponding information through a short-range communication method, such as Bluetooth, and a sound device (not shown) of the user health care device 200 may output the corresponding sound effect. Alternatively, in this case, the SSMT may include a separate sound device (not shown) to output sound effects under the control of the control layer 320.

The sensing layer 330 includes sensor elements (not shown) disposed on the front surface thereof in a lattice form to sense a square (a unit of a square having each side measuring 25 cm) stepped on by a user and output the sensed square. The sensor element may be implemented with various materials, for example, a pressure sensor or a pressure-sensitive sensor for sensing a pressure.

The LED layer 350 includes LED elements (not shown) disposed on the front surface thereof in a lattice form to turn on LEDs in relevant areas in an order corresponding to the first exercise rule RUL1.

The third layer 360 is formed of a transparent material, such as an acrylic or silicone film, to prevent the LED layer 3550 from being damaged while transmitting LED light for the user to recognize the LED light.

Although not shown, the SSMT 300 may further include a power supply unit to supply power to the control layer 320, the sensing layer 330, and the LED layer 350. The power supply unit (not shown) may be implemented in the form of a battery or a charger.

The user confirms the order in which the LEDs on the SSMT 300 are turned on, and when all the LEDs are turned off, steps on the corresponding square areas according to the order in which the LEDs are turned on. As described above, the sensing layer 330 senses the square stepped on by the user, and the control layer 320 processes the sensing information to output the coordinate information and the time (sequence) information of the squares stepped on by the user as exercise information of the user.

Referring again to FIGS. 1 and 2, the exercise mat control unit 213 of the user health care device 200 receives the exercise information of the user (S240) and transmits the received exercise information of the user to the information processing unit 212. As described above, the first exercise rule RUL1 may be selected on the basis of user health information.

Hereinafter, the generating of user health information will be described in more detail.

The user health status analysis and evaluation unit 212c analyzes and evaluates information about a health status of a user in personal information PINF.

For example, a Korean Mini-Mental State Examination (K-MMSE), a brain age, a longevity, and a happiness index may be evaluated.

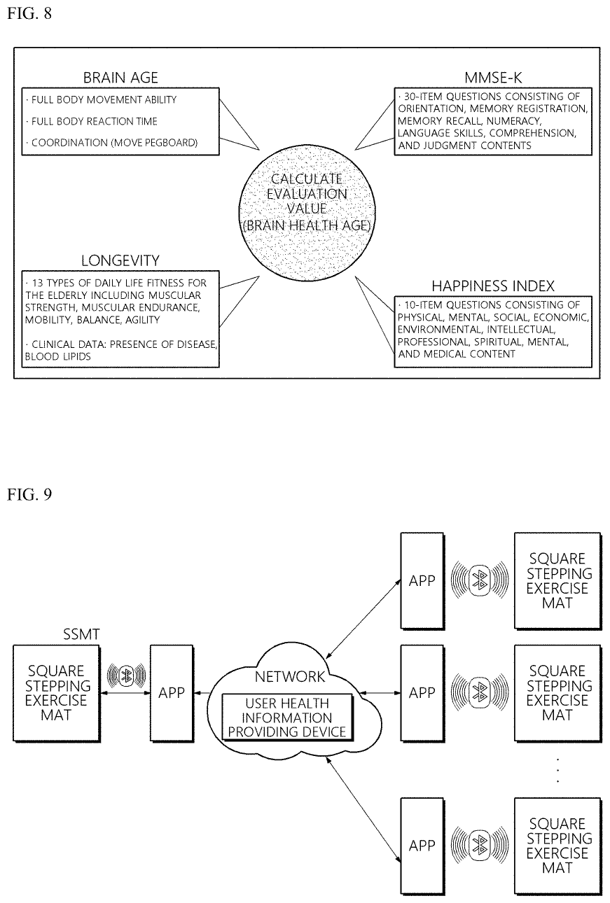

FIG. 8 is a diagram illustrating a method of analyzing and evaluating a health status of a user according to an embodiment of the present invention. Referring to FIGS. 1, 2, and 8, the user health status analysis and evaluation unit 212c may analyze and evaluate a health status of a user on the basis of the K-MMSE, the brain age, the longevity, the happiness index, etc. to calculate a brain age in an index or level that may reflect a brain status and physical status.

For example, through the K-MMSE, orientation (an ability to correctly recognize time, place, situation, environment, etc.), memory, and language ability of the user may be evaluated. The K-MMSE has a total score of 30 points including five points for the orientation to time, five points for the orientation to place, three points for the memory registration ability, five points for the attention and calculation ability, three points for the memory recall, eight points for the language ability, and one point for the spatiotemporal construction ability. The brain age of a user may be calculated by a certain estimating equation by measuring the full body movement ability, coordination ability, full body reaction time, and the like.

Referring again to FIGS. 1 and 2, the user health status analysis and evaluation unit 212c performs analysis and evaluation on the health status of the user through the above method and calculates a corresponding result (a brain health age) (S250). The user health information generation and update unit 212b generates or updates health information of the user on the basis of the brain health age and exercise information TINF of the user (S260).

For example, health information of the user may be obtained by representing the health of the user into an index or level on the basis of the brain health age and then reflecting exercise information in the health information to indicate a more accurate health status. For example, the cognitive function of a user may be classified as level 2 on the basis of the brain health age of the user, but when mistakes occur five times in the first exercise rule RUL1 related to the cognitive function in the SSMT 300, the cognitive function of the user may be adjusted to level three such that health information is generated.

In addition, the user health information generation and update unit 212b may correct the health information of the user by reflecting the health status of the user after the user executes the first exercise rule RUL1 selected on the basis of the calculated health information of the user.

For example, when the health status improved during the first exercise rule RUL1 being executed for two weeks does not have a significant improvement after two weeks elapse, the change period of the exercise rule for the user may be corrected from one month, which is an original setting, to two weeks. The generated health information of the user is transmitted to the exercise rule selection unit 212a to set the first exercise rule RUL1.

Referring continuously to FIGS. 1 and 2, the user health care device 200 includes a user health information storage unit 214 and a user health information output unit 215 to store the health information of the user and provide the user or a health care manager of the user with the health information of the user using a user interface and the like (S270). The user health information storage unit 214 may also store personal information of the user. The user health information output unit 215 may be provided separately or integrated with the user personal information input unit 110.

FIG. 9 is a diagram illustrating a system for providing a health care service in a cloud environment according to an embodiment of the present invention. Referring to FIGS. 1 and 9 the system for providing a health care service according to the embodiment of the present invention described above may be constructed as a cloud system. In this case, each SSMT 300 may receive the first exercise rule RUL1 from a user health information providing device on the Internet through an application (APP) and may transmit exercise information corresponding to a result of the user exercising on the SSMT 300 according to the first exercise rule RUL1 to the user health care device 200 on the Internet.

In this case, the exercise may be performed in the form of a game such that a plurality of users simultaneously proceed with the first exercise rule RUL1 on a plurality of SSMTs 300 and are ranked so that the user's interest may be stimulated and the exercise effect may be maximized. In this case, the user health care device 200 or each application may control the progress of the game.

In addition, although not shown in FIG. 6, health information HINF of a user may be provided to an institution related to senile dementia, for example, the

Ministry of Health and Welfare or a nursing institution or hospital used by the user such that the corresponding information is used to promote the user's health.

As is apparent from the above, according to an embodiment of the present invention, the exercise rule can be directly and visually transmitted to an individual through an LED on a mat, the exercise result about the degree to which the individual has followed the rule in practice can be automatically sensed, and the interest and motivation in exercise of the individual can be more easily induced.

According to an embodiment of the present invention, the sensed exercise result of each individual can be converted into data for each individual and analyzed so that an optimal exercise rule adjusted to the individual can be provided at a later time.

According to an embodiment of the present invention, the present invention is not only used for exercise activity of an individual but also provided in the form of a game that many people join by combining with an already verified exercise program and information technology (IT) so that more active participation of the elderly can be induced.

According to an embodiment of the present invention, a method of performing the square-stepping exercise is determined in consideration of the cognitive function and physical fitness of an individual, etc., to provide a square-stepping exercise method optimized for the individual, thereby efficiently handling management, such as dementia prevention, diagnosis, and improvement.

According to an embodiment of the present invention, the present invention can contribute to the improvement of health of individuals and society.

What is claimed is:

1. A digital mat system for managing and analyzing cognitive and physical abilities of a user, the digital mat system comprising:
a square-stepping exercise mat that provides a sensing result on a square-stepping exercise; and
a user health care device that provides a user with health information,
wherein the square-stepping exercise mat includes: a communication unit configured to receive a first exercise rule optimized for the user with respect to the square-stepping exercise subjected to signal processing; and a pad formed in a rectangular shape having a width of 100 cm and a length of 250 cm and divided into 40 squares each having one side measuring 25 cm,
the pad comprises a first layer and a second layer formed of a material that allows to absorb an internal shock and prevent slipping; a control layer and a sensor layer formed between the first layer and the second layer; a light emitting diode (LED) layer for sequentially illuminating the squares corresponding to the first exercise rule; and a third layer for protecting the LED layer and transmitting LED light, further comprising the control layer configured to control an operation of the sensor layer and the LED layer, and the sensor layer configured to sense the square which the user steps on according to the first exercise rule, and
the user health care device includes a controller, the controller including: a user personal information input unit configured to provide a user interface to receive personal information of the user; an information processing unit configured to select the first exercise rule according to health information of the user generated or updated on the basis of the personal information and the exercise information of the user; and an exercise mat control unit configured to transmit the first exercise rule to the square-stepping exercise mat and receive the exercise information through short-distance communication.

2. The digital mat system of claim 1, wherein the user health care device includes:
a motion sensor module provided at an upper side to detect an exercise motion when the user performs an exercise in an area other than the square-stepping exercise mat and transmit the exercise motion to the controller;
a hologram stage provided at a lower side of the motion sensor module to output a correct exercise posture to the user in a hologram such that the user follows the exercise posture; and
a hologram generating module provided on a lower portion of the hologram stage to generate a hologram on the hologram stage, wherein the controller is installed tin the hologram generating module.

3. The digital mat system of claim 1, wherein the personal information is periodically updated on the basis of the exercise information of the user.

4. The digital mat system of claim 3, wherein the personal information includes information for identifying the user and information about a health status of the user, and
the information about the health status of the user includes at least one of a dementia test result (Korea-Mini Mental State Examination: K-MMSE) of the user, information about a cognitive function of the user, a measurement of daily living fitness of the user, a degree of a risk of the user falling, a brain age of the user, a longevity of the user, and a happiness index of the user.

5. The digital mat system of claim 1, wherein the user health care device is formed using a cloud system, and
the digital mat system further comprises an application for providing a user interface between the user health care device and the square-stepping exercise mat.

6. The digital mat system of claim 1, wherein the information processing unit is configured to:
analyze information about a health status of the user included in the personal information to calculate a brain health age which allows a brain status and a physical status to be reflected; and
reflect the exercise information in the brain health age to generate the health information.
